(19) United States
(12) Patent Application Publication
(10) Pub. No.: US 2010/0021992 A1
(43) Pub. Date: Jan. 28, 2010

(54) ENZYMATIC DECOMPOSITION OF RESIDUAL FORMAMIDE IN POLYMERS

(75) Inventors: Dietmar Haering, Neu-Edingen (DE); Maximilian Angel, Schifferstadt (DE); Hans-Joachim Haehnle, Neustadt (DE); Thomas Friedrich, Darmstadt (DE); Bernhard Hauer, Fussgoenheim (DE); Volker Braig, Weinheim-Luetzelsachsen (DE); Lysander Chrisstoffels, Limburgerhof (DE); Martin Ruebenacker, Altrip (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF AKTIENGESELLSCHAFT, Ludwigshafen (DE)

(21) Appl. No.: 11/917,912

(22) PCT Filed: Jun. 20, 2006
(86) PCT No.: PCT/EP2006/063353

§ 371 Date: Dec. 18, 2007

(30) Foreign Application Priority Data
Jun. 21, 2005 (DE) 10 2005 029 014.0

(51) Int. Cl. C12P 1/00

(52) U.S. Cl. 435/262

(57) ABSTRACT

Method for reducing the formamide content of preparations (P) prepared from N-vinylformamide monomers by polymerization.

The present invention relates to a method for reducing formamide in polymeric preparations which are composed of N-vinylformamide monomers.

### PRIOR ART

Solutions of polymers and copolymers of vinylformamide may comprise formamide as impurity derived from their preparation. The formamide concentrations may typically be in the range from 500 to 20 000 ppm. It is desirable for a number of areas of application, for example cosmetics, to decrease the formamide content. Reduction to levels of less than 500 ppm down to less than 10 ppm is desired, depending on the area of application. It is at the same time essential that the method applied does not change the polymer structure, in particular there must be no generation of amino groups by hydrolysis of the formamide side chains of the polymer.

Attempts to lower the formamide content of polymer solutions by physical deodorization methods such as distillation, steam stripping or gas stripping have proved to be unsuitable.

WO 00/09573 describes the hydrolysis of formamide to formate and ammonia by treating the polymer with acid or base. This procedure does not, however, give satisfactory results because either the hydrolysis is insufficient, or else a significant proportion of the formamide units of the polymer has likewise been hydrolyzed.

Attempts to eliminate formamide by oxidation or reduction have likewise been unsuccessful. As have attempts to convert it chemically with a number of reagents.

In all these cases either there was no conversion of formamide or unwanted side reactions on the polymer were initiated, such as, for example, molecular weight reduction, crosslinking or hydrolysis.

Various enzymes able to hydrolyze formamide are known: Jack bean urease [EC 3.5.1.51 catalyzes with high activity the hydrolysis of urea and with distinctly lower activity the hydrolysis of formamide to formic acid and ammonia (Dixon et al., Can. J. Biochem. 1980, 58 1335-1344; Fishbein, Biochim. Biophys. Acta 1977, 484, 433-442; Blakeley and Zerner, J. Mol. Cat. 1984, 23, 263-292).

Greenwood et al. (FEMS Microbiol. Lett. 1998, 160, 131-135) describe a urease from Methylophilus methylotrophus [EC 3.5.1.5] able to hydrolyze formamide in aqueous solution.

Clarke (Adv. Microbial Physiol. 1970, 4, 179-222) investigated the substrate specificity of aliphatic amidases from Pseudomonas aeruginosa. This entailed investigating various aliphatic amides such as, for example, formamide, acetamide, in an activity assay.

Wyborn et al. (Eur. J. Biochem. 1996, 240, 314-322; Microbiology 1994, 140, 191-195) investigated a formamidase from Methylophilus methylotrophus. A formamide hydrolysis activity assay was carried out during the purification of the enzyme and heterologous expression.

Cornelius et al. (J. General Microbiol., 1981, 125, 367-374) describe two amidases from Alcaligenes eutrophus. The substrate specificity for formamide and other aliphatic amides was investigated in activity assays.

### OBJECT

It was an object of the invention to reduce the formamide content in aqueous polymer preparations under mild conditions such that the polymer structure is not attacked thereby.

### DESCRIPTION OF THE INVENTION

The invention relates to a method for reducing the formamide content of preparations (P) prepared from N-vinylformamide monomers by polymerization, by bringing a hydrolase of enzyme classification E.C. 3.5 into contact with (P).

Hydrolases suitable for the method of the invention are those acting on non-peptidic carbon-nitrogen bonds (E.C. classification 3.5.x.x.).

Preferred among these are the classes of hydrolases which react with linear amides [EC 3.5.1.x] and very particularly preferably the class of ureases [E.C. 3.5.1.5.] and formamidases [EC 3.5.1.49].

Particularly suitable among the ureases is the urease from jack beans, as is a urease from Lactobacillus, especially Lactobacillus fermentum (“acid urease”) and a urease from Bacillus, in particular Bacillus pasteurii.

A particularly suitable formamidase is the formamidase from Methylophilus methylotrophus described at the outset by Wyborn et al.

The enzymes can either be employed in the method of the invention purified in various purity levels or else be used as relatively impure extract, for example as bean meal (jack bean meal; commercially available). The enzyme is preferably employed as solid.

The enzymes can be employed in pure form or as mixtures (“cocktail”) of a plurality of different enzymes.

Depending on the procedure for the method, the enzyme can be either brought into contact with (P) in dissolved form, or the enzyme is applied to a conventional carrier and brought into contact with (P) in immobilized form.

The amount of the hydrolase used in the method of the invention depends on the degree of purity of the enzyme preparation. Typical amounts of enzyme for the method of the invention are from 10 to 10 000 units, preferably 100 to 1000 units, per g of polymer solid. One unit is defined as the amount of enzyme which hydrolyzes 1 μmol of urea per minute at pH=7.5 and 25° C. These amounts of enzyme are only approximate guidelines from which downward and upward deviations are also possible, especially if the reaction conditions, such as temperature and incubation time, are varied. The amount of hydrolase which is optimal for a particular conversion of the invention can easily be established by routine series of experiments.

The method of the invention is applicable to all polymers which comprise N-vinyl-formamide units. Suitable in this connection are both homopolymers and copolymers of N-vinylformamide, and graft copolymers thereof. Examples of possible comonomers suitable for N-vinylformamide are other N-vinylcarboxamides such as N-vinyl-N-methylformamide, N-vinylacetamide, N-vinyl-N-methylacetamide, N-vinyl-N-ethyl-formamide, N-vinyl-N-n-propylformamide, N-vinyl-N-isopropylformamide, N-vinyl N-isobutylformamide, N-vinyl-N-methylpropionamide, N-vinyl-N-butylacetamide and N-vinyl-N-methylpropionamide.

Further comonomers suitable for N-vinylformamide are monoethylenically unsaturated carboxylic acids having 3 to 8 C atoms, and the water-soluble salts of these monomers. This group includes for example acrylic acid, methacrylic acid, dimethylacrylic acid, ethacrylic acid, maleic acid, citraconic acid, methylenemalonic acid, allylacetic acid, vinylacetic acid, crotonic acid, fumaric acid, mesaconic acid and itaconic acid. The monomers from this group which are preferably used are acrylic acid, methacrylic acid, maleic acid or else mixtures of said carboxylic acids, especially mixtures of acrylic acid and methacrylic acid. The unsaturated carboxylic acids can be polymerized both in free form and in partially or completely base-neutralized form, e.g. with sodium hydroxide solution, potassium hydroxide solution, calcium hydroxide or ammonia.

Examples of further suitable comonomers are esters, amides and nitriles of the stated carboxylic acids. The acrylic and methacrylic esters are preferably derived from saturated monohydric alcohols having 1 to 4 carbon atoms or saturated dihydric alcohols comprising 2 to 4 carbon atoms.

Examples of these esters are methyl acrylate, methyl methacrylate, ethyl acrylate, ethyl methacrylate, n-propyl acrylate, n-propyl methacrylate, isopropyl acrylate, isopropyl methacrylate and the esters of acrylic acid and methacrylic acid which are derived from isomeric butanols, as well as hydroxyethyl acrylate, hydroxyethyl methacrylate, hydroxypropyl acrylate, hydroxypropyl methacrylate, hydroxybutyl acrylate, hydroxyisobutyl acrylate and hydroxyisobutyl methacrylate.

Mention should also be made of acrylamide, methacrylamide, N,N-dimethylacrylamide, N-tert-butylacrylamide, acrylonitrile, methacrylonitrile, dimethylaminoethyl acrylate, dimethylaminoethyl methacrylate, diethylaminoethyl acrylate, diethylaminoethyl methacrylate, and the salts of the last-mentioned monomers with carboxylic acids or mineral acids, and the quaternized products.

It is additionally possible to employ as comonomers for the copolymerization of N-vinylformamide vinyl esters such as vinyl formate, vinyl acetate and vinyl propionate. Also suitable are N-vinylpyrrolidone, N-vinylcaprolactam, 1-vinylimidazole, 2-methyl-1-vinylimidazole and 4-methyl-1-vinylimidazole, vinylsulfonic acid, allylsulfonic acid, methallylsulfonic acid, styrenesulfonic acid, vinylphosphonic acid, allylphosphonic acid and diallyldimethylammonium chloride. It is, of course, also possible to employ mixtures of said monomers.

Units of said comonomers may constitute for example from 1 to 99 mol % of the N-vinylformamide-containing copolymers.

It is moreover possible for homopolymers and copolymers of N-vinylformamide to be modified in such a way that the polymerization is carried out in the presence of compounds which have at least two ethylenically unsaturated nonconjugated double bonds in the molecule. Inclusion of these monomers in the polymerization has the effect of increasing the molecular weight of the polymer.

Particularly suitable examples are alkylenebisacrylamides such as methylenebis-acrylamide and N,N′-acryloylethylenediamine, N,N′-divinylethyleneurea, N,N′-divinyl-propyleneurea, ethylide bis-3-(N-vinylpyrrolidone), N,N′-divinyidiimidazolyl-(2,2′)butane and 1,1′-bis(3,3′-vinylbenzimidazolin-2-one)-1,4-butane. Other suitable crosslinkers are, for example, alkylene glycol di(meth)acrylates such as ethylene glycol diacrylate, ethylene glycol dimethacrylate, tetraethylene glycol diacxylate, tetraethylene glycol dimethacrylate, diethylene glycol acrylate, diethylene glycol methacrylate, aromatic divinyl compounds such as divinylbenzene and divinyltoluene, and vinyl acrylate, allyl acrylate, allyl methacrylate, divinyidioxane, pentaerythritol triallyl ether, and mixtures of the crosslinkers. The crosslinkers are used in amounts of from 0.1 to 10, preferably 1 to 4, % by weight based on the monomers employed in the polymerization.

A further possibility for modification is provided by grafting N-vinylformamide onto other polymers. Graft polymers of this type are prepared by polymerizing N-vinylformamide, if appropriate together with other aforementioned comonomers, in the presence of the grafting base in the manner described previously.

U.S. Pat. No. 5,334,287 describes, for example, the grafting of N-vinylformamide onto natural substances based on saccharides. Suitable as grafting base in this connection are monosaccharides and oligosaccharides such as glucose, fructose, galactose, ribose, mannose, sucrose, lactose and raffinose or polysaccharides such as pectin, algin, chitin, chitosan, heparin, agar, gum arabic, locust bean gum, guar gum, xanthan, dextran and the like, and pentosans such as xylan and araban. Also suitable are native starches from the group of corn starch, potato starch, wheat starch, rice starch, tapioca starch, sago starch, sorghum starch, manioc starch, pea starch or starches having an amylopectin content of at least 80% by weight, such as waxy corn starch or waxy potato starch, enzymatically or hydrolytically degraded starches such as, for example, white and yellow dextrins, and maltodextrins, or else oxidized starches such as, for example, dialdehyde starch.

Finally, also to be mentioned in this series are chemically modified saccharides such as, for example, carboxymethylcellulose.

Further polymers suitable as grafting base are those comprising alkylene oxide units, in particular homopolymers and copolymers of C2- to C4-alkylene oxides which are obtainable by polymerization of ethylene oxide, propylene oxide, n-butylene oxide, isobutylene oxide or tetrahydrofuran. Such polyalkylene oxides are described in DE-A-19515943. These polymers may also be addition products of C2- to C4-alkylene oxides with predominantly long-chain alcohols, phenols, carboxylic acids and amines. DE-A-19526626 discloses corresponding graft polymers on polymers which comprise units of vinyl esters of saturated C1- to C4-carboxylic acids, such as vinyl formate, vinyl acetate, vinyl propionate and vinyl n-butyrate, and/or vinyl alcohol units.

The polymers comprising N-vinylformamide units are prepared by the known processes of solution, precipitation, suspension or emulsion polymerization with use of compounds which remain free radicals under the polymerization conditions. The polymerization temperatures are normally in the range of, for example, 30 to 200, preferably 40 to 110° C. Suitable initiators are, for example, azo and peroxy compounds, and the usual redox initiator systems such as combinations of hydrogen peroxide and hydrazine. These systems may if appropriate additionally comprise small amounts of a heavy metal salt. It is preferred to use as polymerization initiator water-soluble azo compounds such as 2,2′-azobis(2-methylpropionamidine) dihydrochloride, 2,2′-azobis(4-methoxy-2,4-dimethylcaleronitrile) and 2,2′-azobis(2-methyl-N-phenylpropionamidine) dihydrochloride. Polymers of low K value are prepared by expediently carrying out the polymerization in the presence of regulators such as, for example, mercapto compounds, allyl compounds, aldehydes or hydrazine.

The polymers obtainable in this way have K values of from 10 to 300, preferably 30 to 250. The K values are determined by the method of H. Fikentscher, Cellulose-Chemie, Volume 13, 58 to 64 and 71 to 74 (1932), in 5% strength aqueous sodium chloride solution at pH 7 at a temperature of 25° C. and with a polymer concentration of 0.1% by weight.

The formamide concentration in the polymers prepared in this way is in the range of 10-100 000 ppm. Typical values are 5000-10 000 ppm. The method of the invention now allows this formamide content to be markedly reduced.

The reaction temperature at which the hydrolase is brought into contact with (P) can be chosen in wide ranges from about 5° C. up to 100° C. The temperature preferably selected is one at which the hydrolase used has a high catalytic activity, which is normally the case between 10 and 40° C., preferably between 20 and 35° C. If hydrolases from thermophilic microorganisms or enzymes specifically selected for temperature resistance are employed, however, the reaction temperature may also be distinctly higher.

The pH range for the method of the invention is normally between 3 and 10, preferably between 4 and 8. The optimal range depends on the pH stability of the hydrolase used.

The reaction time depends greatly on the chosen amount of enzyme, on the temperature and on the concentration of the polymer in (P), and also on the desired “residual formamide content”. The reaction time is normally in the range from a few hours up to a few days, preferably between 5 and 24 hours.

In the method of the invention, the polymeric preparation is normally present in an aqueous dispersant or solvent, preferably in a buffered solvent. (P) can be in the form either of a dispersion or, preferably, of a molecular solution. The amount of polymer in the preparation (P) can be adjusted within wide ranges, from about 1 up to 99% by weight, preferably from 5 to 50% by weight, particularly preferably from 10 to 30% by weight of polymer.

The method of the invention can be carried out discontinuously or continuously. In a discontinuous (batch) procedure, the enzyme can be added immediately after the polymerization or else after isolation and, if appropriate, purification of the polymer. After the formamide has been degraded by the hydrolase, the enzyme can be removed from the reaction medium, depending on the purpose of use. However, the enzyme can also be left in the reaction medium and be inactivated only if desired, for example by heating or by acidification of the medium.

For a continuous procedure in a preferred embodiment of the invention, a supported enzyme is used for bringing into contact with the polymeric preparation (P). For example, the supported hydrolase can be packed into a column through which the (P) is pumped under the desired reaction conditions. For specific applications it is also possible for cascades of enzyme reactors to be connected in series in order to achieve particularly efficient reduction in formamide.

It is possible with the method of the invention to decrease drastically the formamide content in polymeric preparations prepared from N-vinylformamide by polymerization, and thus to achieve residual formamide contents of less than 50, preferably less than 20, in particular less than 10 ppm.

### EXPERIMENTAL SECTION

The enzyme activity units refer, unless indicated otherwise, to the hydrolysis of urea to ammonia.

The formamide content in the aqueous solution was quantified by HPLC.

### Example 1

### Degradation of Residual Formamide with Extract of Jack Bean Meal

Jack bean meal (supplied by Sigma) was extracted with water, clarified by centrifugation and concentrated by ultrafiltration. An extract containing 1101 U of urease per ml was obtained.

Various amounts of this urease extract were added to a 16 wt % solution of a poly(vinylformamide) in water (total mass of the batch 50 g) and stirred at 40° C. and pH 7.0. The enzyme was denatured by acidification in order to stop the reaction.

| | | Residual formamide |
|---|---|---|
| Amount of enzyme [kU] | Reaction time | content [ppm] |
| none | 0 h | 1600 |
| none | 24 h | 1700 |
| 5 | 6 h | 990 |
| 5 | 24 h | 420 |
| 10 | 6 h | 600 |
| 10 | 24 h | 80 |
| 15 | 6 h | 320 |
| 15 | 24 h | 40 |

### Example 2

### Degradation of Residual Formamide with Lyophilizate of Jack Bean Meal

The extract described in Example 1 was freeze dried. A urease-containing powder with an activity of 17926 U/g was obtained.

Various amounts of this urease powder were added to 50 g of a 16 wt % solution of a poly(vinylformamide) in water and stirred at 40° C. and pH 7.0. The enzyme was denatured by acidification in order to stop the reaction.

| | | Residual formamide |
|---|---|---|
| Amount of enzyme [kU] | Reaction time | content [ppm] |
| none | 24 h | 1700 |
| 1.25 | 24 h | 790 |
| 2.5 | 24 h | 390 |
| 5 | 24 h | 100 |
| 10 | 24 h | 10 |
| 15 | 6 h | 230 |
| 15 | 24 h | <10 |

### Example 3

### Degradation of Residual Formamide with Extract of Soybean Meal

Soybean meal (supplied by Sigma) was extracted with water, clarified by centrifugation and concentrated by ultrafiltration. An extract containing 292 U of urease per ml was obtained.

Various amounts of this urease extract were added to a 16 wt % solution of a poly(vinylformamide) in water (total mass of the batch 50 g) and stirred at 40° C. and pH 7.0. The enzyme was denatured by acidification in order to stop the reaction.

| | | Residual content of |
|---|---|---|
| Amount of enzyme [kU] | Reaction time | formamide [ppm] |
| none | 24 h | 1700 |
| 5 | 6 h | 1220 |
| 5 | 24 h | 610 |
| 7.3 | 6 h | 1150 |

### Example 4

### Degradation of Residual Formamide with Commercial Urease

A purified urease from sword beans is commercially available from Fluka (order No. 94282). The activity for hydrolysis of urea is about 35 units per mg of protein.

Various amounts of this urease extract were added to a 16 wt % solution of a poly(vinylformamide) in water (total mass of the batch 50 g) and stirred at 40° C. and pH 7.0. The enzyme was denatured by acidification in order to stop the reaction.

| | Residual formamide |
|---|---|
| Amount of enzyme | content [ppm] |
| none | 1500 |
| 28.5 mg (1 kU) | 570 |
| 57 mg (2 kU) | 170 |
| 114 ng (4 kU) | 14 |
| 171 mg (6 kU) | <10 |
| 229 mg (8 kU) | <10 |

### Example 5

### Degradation of Residual Formamide with Commercial Urease with a Different Temperature and Time and Higher Polymer Concentration than in Example 4

A purified urease from sword beans is commercially available from Fluka (order No. 94282). The activity for hydrolysis of urea is about 35 units per mg of protein.

Various amounts of this urease extract were added to a 32 wt % solution of a poly(vinylformamide) in water (total mass of the batch 150 g) and stirred at 40° C. and pH 7.0. The enzyme was denatured by acidification in order to stop the reaction.

| | Residual | Residual | Residual |
|---|---|---|---|
| Amount | Formamide | formamide | formamide |
| of | content [ppm] | content [ppm] | content [ppm] |
| enzyme | after 8 h | after 24 h | after 72 h |
| 0 | — | 3400 | — |
| 7.5 kU | — | — | 220 |
| 15 KU | 1170 | 270 | 70 |
| 30 kU | 610 | 25 | 20 |
| 60 kU | 140 | 40 | — |

### Example 6

### Degradation of Residual Formamide with Commercial Urease from Bacillus Pasteurii

A purified urease from Bacillus pasteurii is commercially available from Sigma (order No. U7127). The activity for hydrolysis of urea is about 197 units per mg of protein.

Various amounts of this urease extract were added to a 16 wt % solution of a poly(vinylformamide) in water (total mass of the batch 20 g) and stirred at 40° C. and pH 7.5. The enzyme was denatured by acidification in order to stop the reaction.

| | Residual formamide |
|---|---|
| Amount of enzyme | content [ppm] |
| none | 1200 |
| 10 kU | 400 |
| 20 kU | 120 |

We claim:

1. A method for reducing the formamide content of preparations (P) prepared from N-vinylformamide monomers by polymerization, by bringing a hydrolase of enzyme classification E.C. 3.5 into contact with (P).

2. The method according to claim 1, wherein the hydrolase possesses the enzyme classification E.C. 3.5.1.

3. The method according to claim 2, wherein the hydrolase possesses the enzyme classification E.C. 3.5.1.5.

4. The method according to claim 1, wherein (P) and hydrolase are brought into contact at a pH of between 3-10.

5. The method according to claim 1, wherein (P) and hydrolase are brought into contact at a temperature of between 5 and 100° C.

6. The method according to claim 1, wherein (P) is in the form of an aqueous solution.

7. The method according to claim 1, wherein (P) is a homopolymer of N-vinylformamide.

8. The method according to claim 1, wherein the method is carried out continuously.

9. The method according to claim 1, wherein the formamide content is decreased to below 50 ppm.
